(19) United States
(12) Patent Application Publication
(10) Pub. No.: US 2019/0133146 A1
(43) Pub. Date: May 9, 2019

(54) METHOD FOR ENHANCING POSTPROCESSING CONTENT OF BENEFICIAL COMPOUNDS IN BEVERAGES NATURALLY CONTAINING SAME

(71) Applicants: LORETTA ZAPP, BOULDER, CO (US); THOMAS J. SLAGA, AUSTIN, TX (US); JIFU ZHAU, OLYMPIA, WA (US); MARK LANGE, ROUGEMONT, NC (US)

(72) Inventors: LORETTA ZAPP, BOULDER, CO (US); THOMAS J. SLAGA, AUSTIN, TX (US); JIFU ZHAU, OLYMPIA, WA (US); MARK LANGE, ROUGEMONT, NC (US)

(21) Appl. No.: 16/118,160

(22) Filed: Aug. 30, 2018

Related U.S. Application Data
(63) Continuation-in-part of application No. 14/486,700, filed on Sep. 15, 2014, now Pat. No. 10,080,376.
Application No. 14/486,700 is a continuation of application No. 13/741,663, filed on Jan. 15, 2013, now abandoned.
Application No. 13/741,663 is a continuation of application No. 12/749,163, filed on Mar. 29, 2010, now Pat. No. 8,357,419.
Application No. 12/749,163 is a continuation of application No. 10/962,758, filed on Oct. 12, 2004, now Pat. No. 7,713,566.
Application No. 10/962,758 is a continuation-in-part of application No. 10/493,780, filed on Aug. 13, 2004, now abandoned.
Application No. 10/493,780 is a continuation-in-part of application No. 10/001,928, filed on Oct. 31, 2001, now Pat. No. 6,723,368.
Application No. 10/001,928 is a continuation-in-part of application No. 09/843,543, filed on Apr. 25, 2001, now abandoned.
Application No. 09/843,543 is a continuation-in-part of application No. 09/481,279, filed on Jan. 11, 2000, now Pat. No. 6,669,979.
Application No. 09/481,279 is a continuation-in-part of application No. 09/468,560, filed on Dec. 21, 1999, now abandoned.

(51) Int. Cl. A23F 5/10; A23F 5/00; A23F 5/24; A23F 5/26; A61K 36/74; A23F 5/14; A23F 5/02; A23F 5/04

(52) U.S. Cl. CPC A23F 5/10; A23F 5/00; A23F 5/24; A23F 5/04; A61K 36/74; A23F 5/14; A23F 5/02; A23F 5/267

(57) ABSTRACT

A process for enhancing polyphenolics content of beverages brewed from polyphenolic containing, processed beverage substrate by pre-soaking substrate (coffee beans, for example) before roasting and then quenching the substrate after processing with the liquid in which the substrate was first “pre-soaked.” Beverages produced from the treated substrate exhibit substantially increased polyphenolics content, when compared to conventionally processed beverage substrate of the same nature and processing.

### CITATION TO PRIOR APPLICATIONS

The present application is a continuation-in-part of U.S. application Ser. No. 14/486,700, filed Sep. 15, 2014, which is a continuation of U.S. application Ser. No. 13/741,663, filed Jan. 15, 2013, which is a continuation of U.S. application Ser. No. 12/749,163, filed Mar. 29, 2010, which now U.S. Pat. No. 8,357,419, which is a continuation of U.S. application Ser. No. 10/962,758, filed Oct. 12, 2004, now U.S. Pat. No. 7,713,566, which in turn is a continuation-in-part of U.S. application Ser. No. 10/493,780, which was filed Apr. 26, 2004, as the National Stage Application of PCT/US02/35053. PCT/US02/35053, filed Oct. 31, 2002 claims priority to U.S. application Ser. No. 10/001,928, filed Oct. 31, 2001, now U.S. Pat. No. 6,723,368, issued Apr. 20, 2004, which was a continuation-in-part with respect to U.S. application Ser. No. 09/843,543, filed Apr. 25, 2001, which was a continuation-in-part of U.S. application Ser. No. 09/481,279, filed Jan. 11, 2000, which, in turn, was a continuation-in-part of U.S. application Ser. No. 09/468,560, filed on Dec. 21, 1999, from all of which priority is claimed under 35 U.S.C. § 120.

### BACKGROUND OF THE INVENTION

### 1. Field of the Invention

The present invention relates to conventional foods, beverages, and nutritional supplements.

### 2. Background Information

Recent research indicates that polyphenols in fruits, vegetables, common beverages and plants possess the capacity for diversified, beneficial pharmacological activities. It is widely accepted that these compounds, recently dubbed “vitamin P”, possess a wide range of beneficial pharmacological activities which include stabilizing capillary wall tissues, quenching free radicals, maintaining proper permeability and flexibility of capillaries, and preventing cardiovascular diseases. (C. Castelluccio, et. al. FEBS Letters 368 (1995) 188-192). Numerous studies have also shown that most plant polyphenols possess cancer preventive capacity because of their profound antioxidant activity.

It is, of course, well-known that coffee contains caffeine. However, a lesser-known fact is that coffee contains potentially highly beneficial condensed tannin and polyphenolic acids.

Phenolic acids in coffee are mainly esters of quinic acid with different amount of caffeyl groups attached to its different positions. The phenolic acids present in coffee such as chlorogenic acid, caffeic acid, and para-coumaric acid have been shown to exert cancer preventive activities in animal models. Chlorogenic acid has also been found to inhibit methylazoxymethanol-induced large intestinal tumors in hamster.

Chlorogenic acid, which is the main phenolic acid in coffee, is able to protect the gastric mucosa against irritations, and, therefore, improves the digestibility of foods, beverages and medicaments. The improved digestibility is expressed through a much-reduced systemic acid secretion (such as causes heartburn, etc.), which has been found to be directly dependent on an increased level of chlorogenic acid content in raw green coffee beans.

Normally the natural chlorogenic acid content of green coffee is reduced by approximately 40 to 80% during conventional roasting process. Analysis by the present inventor indicates that green coffee beans which initially contain 8% phenolic acids contain, respectively, 2% phenolic acids when light roasted, 1% when medium roasted, and less than 0.5% when dark roasted. This clearly represents a significant loss of beneficial compounds. Thus, the use of a roasting process which is designed to preserve the polyphenols normally lost through the roasting process will result in a product which has concentrations of phenolic compounds in greater quantities than currently marketed coffee beverages.

The resulting beverage will also be a source of diterpenes which have detoxification properties in humans, as well as other beneficial compounds such as triterpenes.

Furthermore, along with the potential health benefits achieved in the beverage substrate with the said process, antioxidants also have the ability to increase the duration and freshness of conventional foods and beverages. This fact has been known and practiced for many decades in the food industry. Antioxidants reduce the oxidation potential of the constituents found in plant derived foods and beverages which can provide an extended shelf life and stability of the final product.

### BRIEF DESCRIPTION OF THE DRAWINGS

FIG. 1 is a chart representing acrylamide results in ten commercial roasts, 5 with conventional water quench (gray) and five utilizing the HealtyRoast® quench (black).

FIG. 2 is a chart representing acrylamide relative roast temperature using water quench (gray) versus HealthyRoast quench (black).

FIG. 3 is a chart representing acrylamide relative to degree of roast (measured by Agtron) using water quench (gray) versus HealthyRoast quench (black).

### DETAILED DESCRIPTION OF THE PREFERRED EMBODIMENT

Extensive research by the present inventors produced a finding that post-processing chlorogenic acid content in particular, and total polyphenol content in general, can be substantially enhanced for brewed coffee through a remarkably simple process. The same is true of other brewed beverages the counterpart substrates of which are known to have a significant polyphenolic constituent. Therefore, while the predominant discussion in this specification focuses on coffee, it must be understood that similar results can be obtained through practice of the methods of the present invention in the context of producing beverages from other materials which naturally contain polyphenolic acids (teas, for example).

The present inventors have discovered that, if applied in the manner prescribed herein, the remarkably simple process of soaking coffee beans in plain water prior to roasting, and, after roasting, “quenching” the beans with a portion of the pre-soak liquid (the soaking water, plus the polyphenols released into the water) will substantially enhance the post-roasting polyphenol content of coffee beans. This represents yet another significant leap forward in the present inventors' work in optimizing the post-processing polyphenol content of coffee as a means for delivering health-enhancing agents to consumers in a most non-intrusive and cost effective manner.

The process of the present invention, when compared with earlier, related processes developed by the present inventors, not only provides a substantial health benefits potential, but permits such benefits to be realized, and the product which carries the benefits to be distributed and sold, with no market or distribution related impediments or inconveniences. This is true, in part, because, unlike some of the referenced prior processes (the subject of first parent application relating to this continuation application) the process for spiking polyphenolics pursuant to the present invention, at least in the case of coffee, takes place at the commercial, roasting stage, rather than at the retail sales level and is, therefore, completely transparent to the end consumer.

Illustrative examples of processes of the present invention follow. It should be understood, of course, that commercial processing according to the present invention will take place on much larger scales than the illustrative examples provided, with proportional increases in the respective constituents (coffee beans, water, pre-soak liquid used for quenching, etc) for larger batches. The first described example is presently believed to be the optimal process for maximizing polyphenol content in coffee beans and ultimately, therefore, in brewed coffee.

According to the most economical version of the present invention, raw, green coffee beans are “pre-soaked” in water as described in more detail hereafter, and a portion (approximately 10% to 20%) of the same water is later used to quench the same beans immediately after roasting. However, as shown below, variations of the same invention involve pre-soaking green coffee beans, roasting other beans, and quenching the roasted beans with the solution from soaking the first, non-roasted beans. These later methods yield end products of even greater phenolics content.

The level of polyphenols, such as chlorogenic acid, in the presoak liquid depends on the variation in the water temperature of soak and the length or duration of the soak. It will typically range from 15%-30% with the optimum level achieved between 25%-28% total chlorogenic acid. Additionally, an increase in the level of polyp he no Is in the presoak liquid quite possibility could be achieved by concentrating the presoak liquid after filtration from the beans. This result was achieved in the lab through sonication and could be achieved in the production process through familiar unit operations such as evaporation. An increase in the concentration of polyphenols in the presoak liquid could have a significant impact on the level of polyphenols achieved after quenching.

### Example 1

Raw green coffee beans are pre-soaked in water for 3 hours at 75 deg C. 1000 grams of green beans soaked in 2000 mls of water. 1000 mls of pre-soak solution (water used for pre-soaking) was retained after beans are removed from water for roasting. 650 grams of pre-soaked green beans are roasted in a traditional coffee roaster with temperature starting at 350 deg F. and increasing to 430 deg F. over a period of approximately 15-18 minutes.

At the conclusion of the roast, the beans are dropped into a container and immediately quenched with 150 mls of the pre-soak solution that contains 23% total chlorogenic acid. The container is agitated slightly to evenly distribute the presoak liquid onto the roasted beans and ensure uptake of the liquid into the bean cell wall. It should be noted the quench step does not produce a roasted bean that is saturated only slightly coated with the presoak liquid. The roasted beans are air-dried and the roasted beans are then ground to a powder and brewed with hot water to produce a coffee beverage.

Chemical analysis showed that the new beverage contains over 20%-65% of the pre-roasted phenolic acid content, specifically representing chlorogenic acid content at 40% 150% (depending on degree of roast—bigger increase with darker roast) over that in traditional roasted coffee of a similar roast color.

### Example 2

Raw green coffee beans are pre-soaked in water for 3 hours at 80 deg C. 1000 grams of green beans soaked in 2000 mls of water. During the presoak step the beans are completely submersed in water and the soak tank is slightly agitated. 1000 mls of pre-soak solution was obtained Pre-soak water with a chlorogenic acid content of 25% is collected for later quenching step. 1400 grams of regular green beans are roasted in a traditional manner. Upon completion of the roast while the beans are still very hot (>400 deg F.) the beans are split into a control and an experimental group and subsequently quenched with either 150 mls of water (control) or 150 mls of the pre-soak solution that has been previously collected from green beans. Quenching consists of atomizing the presoak liquid onto the bean in an even dispersed manner to achieve a bean contact surface area of >90%. (experimental). The green beans used to create the pre-soak quenching solution are not the beans that are use in the roasting. The roasted beans are air-dried then ground to a powder and brewed with hot water to produce a coffee beverage.

Chemical analysis showed that the new beverage contains over 20%-70% of phenolic acid content, representing a 40%-200% chlorogenic acid content over that of the control of the same roast.

### Example 3

Raw green coffee beans are pre-soaked in water for 3 hours at 80 deg C. 1000 grams of green beans soaked in 2000 mls. of water. 1000 mls of pre-soak solution was obtained. Pre-soak water is collected for later quenching step. A portion of the pre-soak water is collected and freeze dried to be used as a fortifying ingredient in the pre-soak quench. 1400 grams of regular green beans are roasted in a traditional manner. Upon completion of the roast the beans are split into a control and an experimental group and subsequently quenched with either 150 mls of water (control) or 150 mls of the pre-soak which has been fortified with 10 grams of freeze dried pre-soak. All pre-soak solution has been previously collected and/or collected and freeze dried from green beans. (experimental). The green beans used to create the pre-soak quenching solution are not the beans that are use in the roasting. The roasted beans are then air dried, ground to a powder and brewed with hot water to produce a coffee beverage.

Chemical analysis showed that the new beverage contains over 120% of phenolic acids, representing approx 250% of chlorogenic acid content of conventionally processed coffee.

### Example 4

Raw green coffee beans are pre-soaked in water for 4 hours at 80 deg C. 1000 grams of green beans soaked in 2000 mls of water. The presoak liquid was decanted and separated from the beans. A vacuum of −20 inches Hg was applied to the soaked beans to remove any additional high polyphenol liquid engaged onto the bean. 100 mls of pre-soak liquid along with an additional 100 mls of vacuum filtrate were collected and used as the quench water. The total chlorogenic acid content of the quench liquid was 28%. 1400 grams of regular green beans are roasted in a traditional manner. Upon completion of the roast while the beans are still very hot (>400 deg F.) the beans are split into a control and an experimental group and subsequently quenched with either 150 mls of water (control) or 150 mls of the pre-soak solution that has been previously collected from green beans. Quenching consists of atomizing the presoak liquid onto the bean in an even dispersed manner to achieve a bean contact surface area of >90%. The green beans used to create the pre-soak quenching solution are not the beans that are use in the roasting. The roasted beans are air-dried then ground to a powder and brewed with hot water to produce a coffee beverage.

Chemical analysis showed that the new beverage contains over 80% of phenolic acid content, representing a 200% chlorogenic acid content over that of the control of the same roast.

Studies completed by a team of scientists at the University of California, Davis lead by Shibamoto (Shibamoto, et. al., J. Agric. Food Chem., Vol 48, No. 11, 2000) indicate there are a number of volatile chemicals, specifically Heterocyclic compounds formed during conventional brewing processes. These compounds including thiophenes, thiazoles, pyrroles, pyrazines and furans, to name a few, have demonstrated some medicinal activities as well. Therefore it may be possible during the quench step of the process to reclaim these theoretically potent constituents with slight modifications in process equipment.

### Example 5

Raw green coffee beans are pre-soaked in water for 3 hours at 80 deg C. 1000 grams of green beans soaked in 2000 mls of water. The presoak liquid was decanted and separated from the beans. 1000 mls of Pre-soak liquid was collected and used as the quench water. The total chlorogenic acid content of the quench liquid was 24%. 1400 grams of regular green beans are roasted in a traditional manner. Upon completion of the roast while the beans are still very hot (>400 deg F.) the beans are split into a control and an experimental group and subsequently quenched with either 150 mls of water (control) or 150 mls of the pre-soak solution that has been previously collected from green beans. In this example quenching consists of atomizing the presoak liquid onto the bean in an even dispersed manner to achieve a bean contact surface area of >90% in an enclosed fluid bed where no steam is allowed to escape throughout the quenching and drying process. The vent vapors are condensed in a reflux chamber and reintroduced back into the fluid bed. The green beans used to create the pre-soak quenching solution are not the beans that are use in the roasting. When the roasted beans are fully air-dried and cooled they are ground to a powder and brewed with hot water to produce a coffee beverage.

HPLC analysis showed that the new beverage contains over 80% of phenolic acid content, representing a 200% chlorogenic acid content over that of the control of the same roast. Further analysis from UV absorbance indicated a presence of additional antioxidant compounds that may have been captured from the volatile vapors further research needs to be conducted to characterize these compounds and determine their antioxidant activities.

The powder from the preceding examples can be sold as coffee powder for brewing, instant coffee, or can be brewed and sold as a ready-to-drink coffee beverage. The resulting product can be taken as a food or functional food by a human or other mammal, orally.

### Example 6

Raw green coffee beans are pre-soaked in water for 4 hours at 95 deg C. 34 kilograms of green beans soaked in 72 liters of water. Drain off the presoak liquid. While the presoak liquid is still hot, add to the presoak liquid 2.55 kilograms of green coffee bean extract purified to greater than 60% total polyphenolic acids, or other water soluble antioxidant based extract with a similar concentration. Allow mixture to agitate for 30 minutes at 60 deg. C. This presoak liquid will now be a concentrated polyphenol solution containing greater than 8% polyphenols.

Roast 250 kilograms of beans at conventional roasting conditions (450 deg F., 7 minutes or roast to achieve desired bean sensory profile). Once roast is complete, quench beans with 32 liters of concentrated presoak liquid for 50 seconds. Allow beans to cool and package accordingly. Note: by discharging the roasted beans immediately after roasting, while still hot, into an appropriate mixer—an example might include a fluidized bed mixer such as a Forberg as commonly known in the field—optimum quench or distribution of the presoak liquid onto the beans can be achieved before the beans are allowed to cool. The optimum temperature for concentrated polyphenol liquid absorption into the bean cell wall is at a bean temperature of greater than 375 deg F. Beans can be ground or packaged as whole beans. It may be necessary, once beans are cool, to slightly agitate them to obtain a free flowing product. The final brewed coffee polyphenol content is 160 mg. per serving.

### Acrylamide Data

### 1. Objective

The objective of this study is to evaluate the potential effects of the HealthyRoast® technology on the acrylamide levels of roasted coffees in comparison to conventionally brewed coffee.

### 2. BACKGROUND

Acrylamide is a hazardous compound formed during the roasting, baking, and frying of foods. Acrylamide is carcinogenic to laboratory rodents and is described by the International Agency for Research of Cancer as a probable carcinogen to humans. The main route of acrylamide formation is represented by the Maillard reaction, which involves the reaction of asparagine with a carbonyl compound.

Even though coffee is not an apparent source of reactive carbonyls, its constituents are susceptible to form many reactions leading to carbonyls that may be involved in acrylamide formation during roasting, e.g. decomposition of sucrose and oxidation of linoleic acid (Kocadagli et al., 2012). In the study by Andrzejewski et. al. (2004) several commercial ground, dry instant, and brewed coffees were tested for acrylamide. The levels found ranged from 45 to 344 ng/g in ground coffees, 172 to 539 ng/g in dry instant coffees, and 6 to 11 ng/mL in brewed coffees. These levels, especially in brewed coffees, are considerably low in comparison to other foods. According to data published by the FDA, while the median level of acrylamide in brewed coffee was 6.5 ng/mL, french fries and chips, crackers and snack foods, nuts and butters exhibited 318 ng/g, 169 ng/g, and 89 ng/g, respectively (Dybing et al., 2005).

Existing strategies to reduce acrylamide content include three basic aspects: modification of raw materials, optimization of processing conditions, and addition of exogenous additives (Jin et al., 2013). For raw materials, the key is to control the content of carbonyl source and asparagine. Choosing a relatively higher storage temperature (Chuda et al., 2003) can reduce the content of reducing sugars in potatoes, thus reduce the acrylamide level in the final product. For processing conditions, an appropriate heating temperature should be set and long-time processing of the foodstuff should be avoided (Mottram et al., 2002). As for exogenous additives, many substances are reported to be effective for the mitigation of acrylamide, including some organic acids (e.g. citric acid, Cook & Taylor, 2005), some amino acids (e.g. glycine, Claeys, De Vleeschouwer, & Hendrickx, 2005), and some mono- and divalent cations (e.g. Na+ or Ca2+, Gökmen & Senyuva, 2007).

In previous studies where antioxidants were applied, both positive and negative results were obtained, indicating that certain antioxidants could reduce, while others could increase the levels of acrylamide (Jim et al., 2013). In the study by Ou et al. (2010) six types of antioxidants were tested in both the acrylamide model system and ASN—GLC model system. Three of the antioxidants were relatively stable (not easily oxidized), and the other three were unstable. Results showed that stable antioxidants (BHA, BHT and TBHQ) had no effect or even promoted acrylamide formation.

The AFS patented HealthyRoast® Technology consists on quenching the roasted coffee beans with a green coffee liquor rich in chlorogenic acids, one of the abundant antioxidants found in coffee. Considering the historical research and previous analysis of HealthyRoast® coffees, we believe this technology could provide roasted coffees with less acrylamide in comparison to those conventionally roasted.

### 3. Material and Methods

3.1. Green Coffee Beans

Green coffee bean “A” was Arabica, organic, crop 2017, from Peru (JAEN-ORG 17-43).

Green coffee bean “B” was Arabica, non-organic, crop 2017, from Peru (030-0644-0449).

The green coffee bean used to obtain the HealthyRoast® liquor was Robusta, non-organic, crop 2017, from Vietnam (0145-4247-18-13 1).

All coffee bags contained 69 kg of beans.

3.2. HealthyRoast® Liquor

For the production of the HeathyRoast® liquid extract, a tank was filled with one bag of green coffee and 80 gallons of water, and heated for 20 hours at 191° F.

3.3. Roasting Cycles

Roasting was conducted with two roasters, a Burns 23R28 and 23RS/ACO. Three 69 lb. bags of green coffee were used for each roasting cycle. Each batch of roasted coffee was quenched with four gallons of liquid. The liquids used to quench the coffees were the HealthyRoast® liquor and potable city water, similar to that used in conventional coffee roasting quench techniques. Conventional water quenching is often used in commercial roasting applications to halt or stop of the roast, allowing for optimum sensory characteristics in the final brewed cup. The beans are then ejected from the roaster approximately two minutes after quenching.

A preliminary study utilizing the commercial scale roasters was completed to determine the potential impact in acrylamide levels with changes in 1) roast temperature, 2) roast time, and 3) impact using the HealthyRoast process relative to a conventional water quench. The results of the preliminary data are presented in Table 1 of Appendix A. From the initial evaluation the two variables which impacted acrylamide with any significance were the roasting temperature or degree of roast and the utilization of HealthyRoast liquor in the quench step instead of water. Overall, the acrylamide level was reduced with an increase in roasting temperature. During a conventional water quench the roast temperatures were varied from 400° F. to 450° F. and the resulting acrylamide levels changed from 400 ppb to 188 ppb. The next parameter evaluated was the impact in roast time; while maintaining a roast temperature of 435° F. roast time was changed from 8.30 minutes to 12 minutes and the results indicate that the final acrylamide levels were constant. The third parameter evaluated was the source of the quench liquor while maintaining similar roast profiles. The roast temperature was targeted at 425° F. for two separate roasts, the first roast was quenched with water and the second was quenched with the HealthyRoast® liquor. Acrylamide levels went from 224 ppb using water and 118 ppb using the HealthyRoast® process. This data was used to set comparison parameters for further evaluation in additional production batches.

Following the preliminary study, six commercial batches were produced on day two; two at 415° F., two at 425° F. and two at 440° F. The source of the quench was modified for each roast temperature from the conventional water quench to the HealthyRoast® liquor while maintaining similar roasting profiles. The following day four additional roasts were produced two at 410° F. using water as the quench liquor and two at 430° F. using HealthyRoast® liquor while again maintaining similar roast profiles. The results from all 10 production roasts are presented in Table 2 of Appendix A.

3.4. Analysis of Roasted Coffee

The final roasting level was confirmed by testing the color of whole and ground coffee beans. An Agtron color analyzer model M-Basic II was used.

The roasted coffee samples were tested for acrylamide at Intertek by LCMSMS and expressed as parts per billion (ppb).

### 4. Results

The results of acrylamide present in the roasted coffee relative to roast temperature from ten commercial scale production batches are provided below.

Production Run 1

| | Water | | |
|---|---|---|---|
| | Quench | HealthyRoast ® | Reduction in |
| Roast Temperature | (acrylamide | Quench | Acrylamide using |
| (deg F.) | ppb) | (acrylamide ppb) | HealthyRoast ® |
| 415 | 287 | 193 | 33% |
| 425 | 182 | 151 | 17% |
| 440 | 130 | 121 | 7% |

Production Run 2

| | | | Reduction in |
|---|---|---|---|
| Roast | | HealthyRoast ® | Acrylamide |
| Temperature | Water Quench | Quench | using |
| (deg F.) | (acrylamide ppb) | (acrylamide ppb) | HealthyRoast ® |
| 410 | 465 | 218 | 53% |
| 430 | 211 | 175 | 17% |

### SUMMARY & CONCLUSIONS

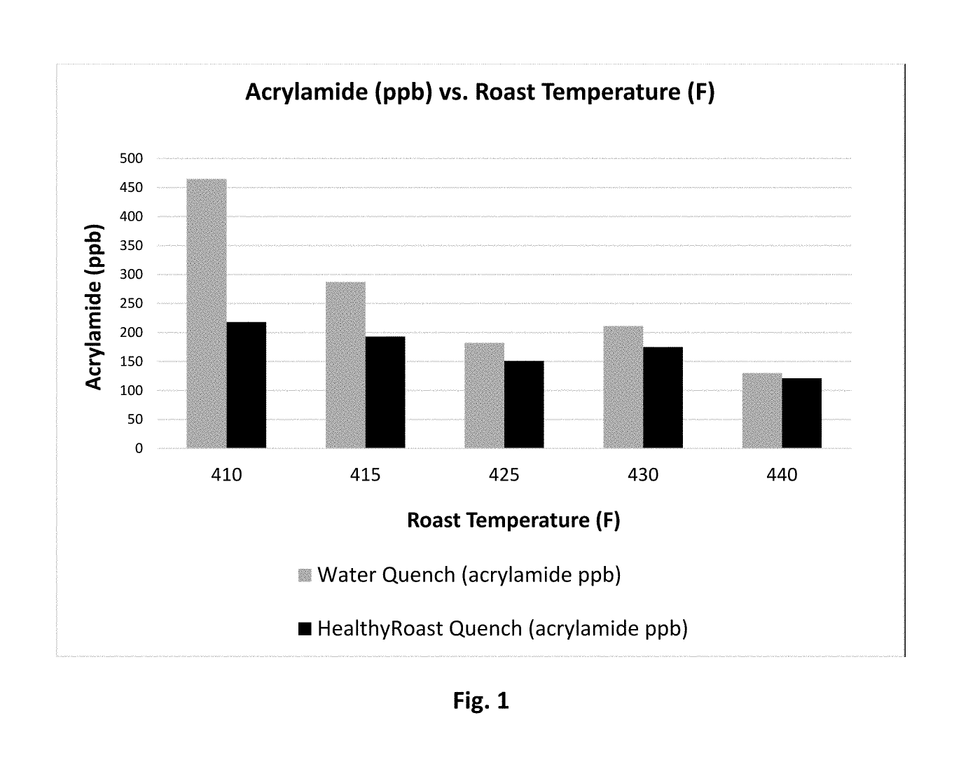

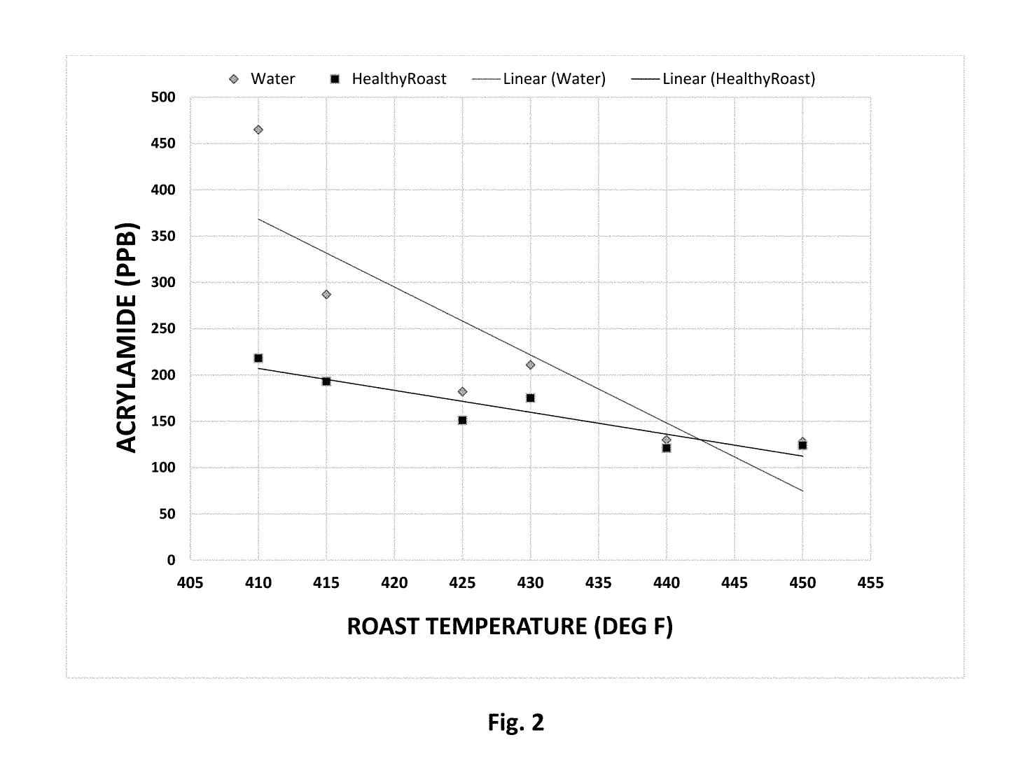

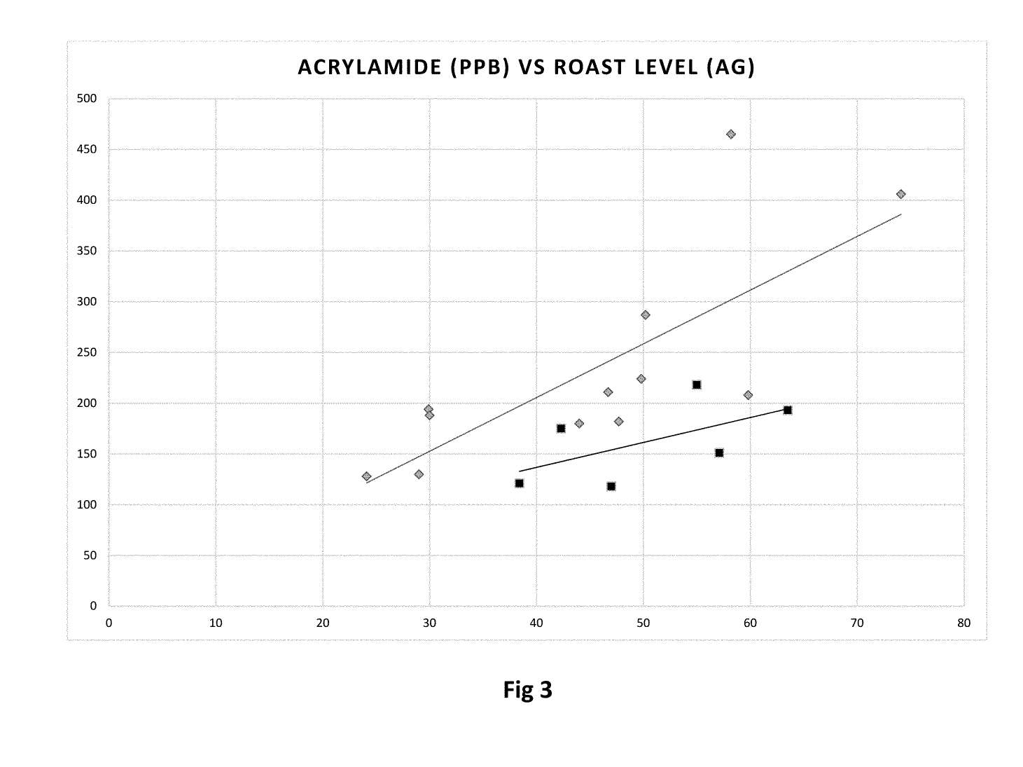

In general, darker roasts and ultimately darker beans exhibited lower acrylamide levels. When roasted at a similar roasting degree, the HealthyRoast® process exhibited less acrylamide in the final roasted beans in comparison to the conventionally brewed coffee (FIG. 1). The results indicate when utilizing the HealthyRoast® process at the lower roast temperatures there is a significant dependent relationship between the amount of reduction in acrylamide and the roast temperature (FIG. 2). The HealthyRoast® process allows roasters the ability to control and reduce acrylamide levels by as much as 50% in light and 30% in the medium roasted coffees. The degree of roast and its impact on final acrylamide level was confirmed by measuring the color of the roasted bean. Typically, the darker the color to darker the roast and the lower the associated Agtron score (Appendix B). The data correlates the degree of roast with the roast temperate (FIG. 3) and the associated reduction in acrylamide when implementing the HealthyRoast® process with the lighter roasted coffees such as the breakfast and house blends.

### APPENDIX A—BATCH DATA

TABLE 1
Bench Top Roasting Results

| Sample | | Green Coffee | | Quench Temp | Quench Time | Quench | Color | Acrylamide |
|---|---|---|---|---|---|---|---|---|
| # | Date | Bean | Roaster | (F.) | (min) | Method | (Ag) | (ppb) |
| 1 | Prelim | B | B | 400 | 13:00 | Water | 74.1 | 406 |
| 2 | Prelim | B | B | 420 | 13:30 | Water | 59.8 | 208 |
| 3 | Prelim | B | B | 450 | 15:00 | Water | 29.9 | 194 |
| 4 | Prelim | B | B | 435 | 12:00 | Water | 44 | 180 |
| 5 | Prelim | B | B | 425 | 12:00 | Water | 49.8 | 224 |
| 6 | Prelim | A | A | 435 | 8:30 | Water | 30 | 188 |
| 7 | Prelim | B | B | 425 | 12.07 | HR | 47 | 118 |

TABLE 2
Production Roast Results

| Sample | | Green Coffee | | Quench Temp | Quench Time | Quench | Color | Acrylamide |
|---|---|---|---|---|---|---|---|---|
| # | Date | Bean | Roaster | (F.) | (min) | Method | (Ag) | (ppb) |
| 8 | Jun. 28, 2018 | A | A | 415 | 12 | Water | 50.2 | 287 |
| 9 | Jun. 21, 2018 | A | A | 425 | 9:21 | Water | 47.7 | 182 |
| 10 | Jun. 21, 2018 | A | A | 440 | 8:30 | Water | 29 | 130 |
| 11 | Jun. 21, 2018 | A | A | 415 | 9:08 | HR | 63.5 | 193 |
| 12 | Jun. 21, 2018 | A | A | 425 | 10:30 | HR | 57.1 | 151 |
| 13 | Jun. 21, 2018 | A | A | 440 | 9:40 | HR | 38.4 | 121 |
| 14 | Jun. 28, 2018 | A | A | 410 | 11:30 | Water | 58.2 | 465 |
| 15 | Jun. 28, 2018 | A | A | 430 | 12:00 | Water | 46.7 | 211 |
| 16 | Jun. 28, 2018 | A | A | 450 | 12:40 | Water | 24.1 | 128 |
| 17 | Jun. 21, 2018 | A | A | 410 | 13:00 | HR | 55 | 218 |
| 18 | Jun. 21, 2018 | A | A | 430 | 13:30 | HR | 42.3 | 175 |

Green Coffee Bean A: Arabica, Organic Peru JAEN-ORG 17-43, 69 kg bags
Green Coffee Bean B: Arabica, Regular Peru 030-0644-0449, 69 kg bags
Roaster A: Burns 23R28 Roaster B: Burns 23RS/ACO WB = whole beans, GB = ground bean.

### APPENDIX B—AGTRON SCALE

| Classification | Gourmet Scale (Ag) | Commercial Scale (Ag) |
|---|---|---|
| Undeveloped | 100 | 75.4 |
| Extremely Light | 95 | 71.7 |
| Very Light | 85 | 64.3 |
| Light | 75 | 56.9 |
| Medium Light | 65 | 49.4 |
| Medium | 55 | 42 |
| Medium Dark | 45 | 34.6 |
| Dark | 35 | 27.1 |
| Very Dark | 25 | 19.7 |

Agtron 90 score similar to cinnamon root
Agtron 30 score represents the nominal development for Italian/French Roast

### CONCLUSION

The preceding examples illustrate that a more healthful polyphenol coffee beverage product can be produced by a very simple variation of conventional coffee roasting methods. In addition, an end product which is healthier and not much more costly than existing coffee powders can be produced, and thereby provide a market and economic benefit to vendors. The present method yields a product which is in no way undesirable from an aesthetic standpoint (taste, aroma, etc. is unaffected). Thus, there is no reason not to, and every reason to, adopt the present coffee roasting processing methods for the well being of consumers.

The potential to increase the antioxidant capacity in the beverage substrate, in this case roasted coffee, provides an end product that has increased stability through a reduction in oxidation potential. Reducing the oxidizing potential of the beverage substrate allows a product to maintain its freshness, taste profile, and other product characteristics for a longer duration than would be achieved through conventional processing methods.

The processes of the present invention represent significant departures from conventional production of roasted coffee products, where green beans are simply roasted and mayor may not be quenched with water, whereas the end product of the present invention achieves a chemical profile of increased amounts of phenolic acids and other beneficial compounds which is different from existing roasted coffee brews. This new process yields more active, more bioavailable, and larger quantities of phenolic compounds than those found in existing roasted coffee brews.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

1. A method of producing a beverage substrate, the method comprising the steps of: selecting a measure of beverage substrate;
immersing said beverage substrate in pre-soak liquid containing water;
removing said beverage substrate from said pre-soak liquid and roasting said beverage substrate; and
quenching said beverage substrate after said roasting with a portion of said post-immersion pre-soak liquid.

2. The method of claim 1 wherein the quenching further includes a water soluble antioxidant-based extract.

3. The method of claim 1 wherein said beverage substrate is coffee beans.

4. A method of producing a beverage substrate, the comprising the steps of:
selecting a first measure of beverage substrate;
immersing said first measure of beverage substrate in a pre-soak liquid containing water;
collecting said pre-soak liquid after said immersing;
roasting a second measure of beverage substrate; and
quenching said second measure of beverage substrate after said roasting with a portion of said post-immersion pre-soak liquid.

5. The method of claim 4 wherein the quenching further includes a water soluble antioxidant-based extract.

6. The method of claim 4 where said first measure of beverage substrate comprises coffee beans.

7. The method of claim 4 wherein said second measure of beverage substrate comprises coffee beans.

8. The method of claim 4 wherein said first measure of beverage substrate and said second measure of beverage substrate comprises coffee beans.

9. A method of producing a beverage substrate, the method comprising the steps of:
selecting a first measure of beverage substrate;
immersing said first measure of beverage substrate in a pre-soak liquid containing water;
collecting the pre-soak liquid after said immersing;
drying a portion of the pre-soak liquid;
roasting a second measure of beverage substrate; and
quenching the second measure of beverage substrate after the roasting with a solution comprising the dried post-immersion pre-soak liquid.

10. The method of claim 9 where said first measure of beverage substrate comprises coffee beans.

11. The method of claim 9 wherein said second measure of beverage substrate comprises coffee beans.

12. The method of claim 9 wherein said first measure of beverage substrate and said second measure of beverage substrate comprises coffee beans.

13. The method of claim 9 where the quenching further includes a water soluble antioxidant-based extract.
